(19) United States
(12) Patent Application Publication
(10) Pub. No.: US 2023/0068517 A1
(43) Pub. Date: Mar. 2, 2023

(54) SURGICAL PLANNING SYSTEMS AND METHODS FOR PREOPERATIVELY ASSESSING CENTER OF ROTATION DATA

(71) Applicant: ARTHREX, INC., Naples, FL (US)

(72) Inventors: Nick METCALFE, Bonita Springs, FL (US); Ryan MEGGER, Chagrin Falls, OH (US); Brandon EVANS, Shaker Heights, OH (US)

(21) Appl. No.: 17/897,579

(22) Filed: Aug. 29, 2022

Related U.S. Application Data
(60) Provisional application No. 63/238,411, filed on Aug. 30, 2021.

(51) Int. Cl. A61B 34/10; G16H 20/40; G16H 70/20; G16H 40/67; G16H 50/50

(52) U.S. Cl. CPC A61B 34/10; G16H 20/40; G16H 70/20; G16H 40/67; G16H 50/50; A61B 2034/102; A61B 2034/105

(57) ABSTRACT

Surgical planning systems and methods are disclosed for planning orthopaedic procedures, including pre-operatively, intra-operatively, and/or post-operatively to create, edit, execute, and/or review surgical plans. The surgical planning systems and methods may be used to preoperatively assess a planned postoperative implant center of rotation relative to a preoperative native anatomy center of rotation. A delta distance between the planned postoperative implant center of rotation and the preoperative native anatomy center of rotation may be utilized to optimize a specific implant center of rotation that is most appropriate for a given patient's anatomy, thereby improving surgical outcomes.

### CROSS-REFERENCE TO RELATED APPLICATIONS

This disclosure claims priority to U.S. Provisional Application No. 63/238,411, which was filed on Aug. 30, 2021 and is incorporated herein by reference in its entirety.

### BACKGROUND

This disclosure is directed to the field of surgical planning, and more particularly to surgical planning systems and methods for planning orthopedic procedures. The surgical planning may include, for example, preoperatively assessing both an anatomical and an implant model center of rotation for selecting the appropriate implant type, size, position, orientation, etc.

Arthroplasty is a type of orthopedic surgical procedure performed to repair or replace diseased joints. Surgeons may desire to establish a surgical plan for preparing a surgical site, selecting an implant, and placing the implant at the surgical site prior to performing arthroplasty in order to improve outcomes. Surgical planning may include capturing an image of the surgical site and determining a position of an implant based on the image.

### SUMMARY

This disclosure relates to improved surgical planning systems and methods.

The surgical planning system and methods of this disclosure may be utilized in some implementations for planning orthopaedic procedures, including pre-operatively, intra-operatively, and/or post-operatively to create, edit, execute, and/or review surgical plans. The surgical planning systems and methods may be utilized for planning and implementing orthopaedic procedures to restore functionality to a joint.

A surgical planning system may include, inter alia, a memory device configured to store computer executable instructions, and a processor operably coupled to the memory device and configured to execute the computer executable instructions. The processor may execute the computer executable instructions to receive an input related to a position of an implant model relative to a bone model within a planning environment, and calculate a deviation between a planned postoperative implant center of rotation of the implant model and a preoperative native anatomy center of rotation of the bone model.

Another exemplary surgical planning system may include, inter alia, a memory device configured to store computer executable instructions, and a processor operably coupled to the memory device and configured to execute the computer executable instructions. The processor may execute a planning environment that includes a display module, a spatial module, and a comparison module. The memory device is configured to store an implant model and a bone model. The bone model includes an identification of a preoperative native anatomy center of rotation. The spatial module is configured to establish a planned postoperative implant center of rotation of an implant of the implant model that is overlayed on the bone model. The comparison module is configured to determine a delta distance between the planned postoperative implant center of rotation and the preoperative native anatomy center of rotation, and the display module is configured to display the delta distance in a display window of a graphical user interface. The planned postoperative implant center of rotation may include an anterior/posterior coordinate, a superior/inferior coordinate, and medial/lateral coordinate that are referenced relative to the preoperative native anatomy center of rotation.

A computer implemented surgical planning method may include, inter alia, receiving a preoperative planning input from a user. The preoperative planning input may include a position of an implant model relative to a bone model of a subject patient, for example. The method may further include identifying a planned postoperative implant center of rotation of an implant of the implant model relative to the bone model, and calculating a delta distance between the planned postoperative implant center of rotation and a preoperative native anatomy center of rotation of the bone model.

The embodiments, examples, and alternatives of the preceding paragraphs, the claims, or the following description and drawings, including any of their various aspects or respective individual features, may be taken independently or in any combination. Features described in connection with one embodiment are applicable to all embodiments, unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 schematically illustrates an exemplary surgical planning system.

FIG. 2 schematically illustrates exemplary aspects of the surgical planning system of FIG. 1.

FIG. 3 schematically illustrates an exemplary user interface of a surgical planning system.

FIG. 4 schematically illustrates another exemplary user interface of a surgical planning system.

FIG. 5 schematically illustrates another exemplary user interface of a surgical planning system.

FIG. 6 schematically illustrates yet another exemplary user interface of a surgical planning system.

FIG. 7 schematically illustrates exemplary databases of a storage system that can be accessed by a surgical planning system.

FIG. 8 schematically illustrates additional aspects of a surgical planning system.

FIG. 9 schematically illustrates an exemplary anatomical makeup classification that can be assigned by a surgical planning system.

FIG. 10 schematically illustrates a method for assessing center of rotation information within a surgical planning system.

### DETAILED DESCRIPTION

This disclosure is directed to improved surgical planning systems and methods for planning orthopaedic procedures, including pre-operatively, intra-operatively, and/or post-operatively to create, edit, execute, and/or review surgical plans. The surgical planning systems and methods may be utilized for planning and implementing orthopaedic procedures to restore functionality to a joint.

In some embodiments, the surgical planning systems and methods may be used to preoperatively assess a planned postoperative implant center of rotation relative to a preoperative native anatomy center of rotation. A delta distance between the planned postoperative implant center of rotation and the preoperative native anatomy center of rotation may be utilized to optimize a specific implant center of rotation that is most appropriate for a given patient's anatomy, thereby improving surgical outcomes. These and other features of this disclosure are discussed in greater detail in the following paragraphs of this detailed description.

A surgical planning system according to an exemplary aspect of this disclosure may include a memory device configured to store computer executable instructions, and a processor operably coupled to the memory device and configured to execute the computer executable instructions. The processor may execute the computer executable instructions to receive an input related to a position of an implant model relative to a bone model within a planning environment, and further to calculate a deviation between a planned postoperative implant center of rotation of the implant model and a preoperative native anatomy center of rotation of the bone model.

In a further implementation, the preoperative native anatomy center of rotation is the native center of rotation about which a joint mechanics of a joint associated with the bone model will revolve.

In a further implementation, the preoperative native anatomy center of rotation is an interpolation of an original, non-deteriorated anatomy of a patient associated with the bone model.

In a further implementation, the planned postoperative implant center of rotation is a value associated with an implant associated with the implant model.

In a further implementation, the implant is a glenosphere implant.

In a further implementation, the implant is a humeral head implant.

In a further embodiment, the deviation is a delta distance representing a length of a vector in a three dimensional space between the planned postoperative implant center of rotation and the preoperative native anatomy center of rotation.

In a further implementation, the processor is further configured to cause the delta distance to be displayed within a user interface of the surgical planning environment.

In a further embodiment, the delta distance is visually indicated by a line extending between the planned postoperative implant center of rotation and the preoperative native anatomy center of rotation.

In a further implementation, the delta distance is visually indicated by a numerical value in a graphic indicator of the user interface.

In a further implementation, the processor is further configured to query a database of the surgical planning system for records having similar center of rotation characteristics.

In a further implementation, the database is an anatomical makeup classification database that stores a plurality of anatomical makeup classifications that characterize anatomical differences and variances within the anatomical differences within a representative patient population.

In a further implementation, each of the plurality of anatomical makeup classifications is a numerical classification of an anatomical makeup of a bone or a joint of the representative patient population.

In a further embodiment, the processor is further configured to command that a user be prompted to assess a probability of a successful surgical outcome based on the records having the similar center of rotation characteristics.

A surgical planning system according to another exemplary aspect of this disclosure may include a memory device configured to store computer executable instructions, and a processor operably coupled to the memory device and configured to execute the computer executable instructions. The processor may execute a planning environment that includes a display module, a spatial module, and a comparison module. The memory device is configured to store an implant model and a bone model. The bone model includes an identification of a preoperative native anatomy center of rotation. The spatial module is configured to establish a planned postoperative implant center of rotation of an implant of the implant model that is overlayed on the bone model. The comparison module is configured to determine a delta distance between the planned postoperative implant center of rotation and the preoperative native anatomy center of rotation, and the display module is configured to display the delta distance in a display window of a graphical user interface. The planned postoperative implant center of rotation may include an anterior/posterior coordinate, a superior/inferior coordinate, and medial/lateral coordinate that are referenced relative to the preoperative native anatomy center of rotation.

In a further implementation, the delta distance is visually indicated by a line extending between the planned postoperative implant center of rotation and the preoperative native anatomy center of rotation.

In a further implementation, the delta distance is visually indicated by a numerical value in the display window.

In a further implementation, the preoperative native anatomy center of rotation is an interpolation of an original, non-deteriorated anatomy of a patient associated with the bone model.

In a further implementation, the planned postoperative implant center of rotation is a value associated with the implant, and the implant is a glenosphere implant or a humeral head implant.

A computer implemented surgical planning method according to yet another exemplary aspect of this disclosure may include receiving a preoperative planning input from a user. The preoperative planning input may include a position of an implant model relative to a bone model of a subject patient, for example. The method may further include identifying a planned postoperative implant center of rotation of an implant of the implant model relative to the bone model, and calculating a delta distance between the planned postoperative implant center of rotation and a preoperative native anatomy center of rotation of the bone model.

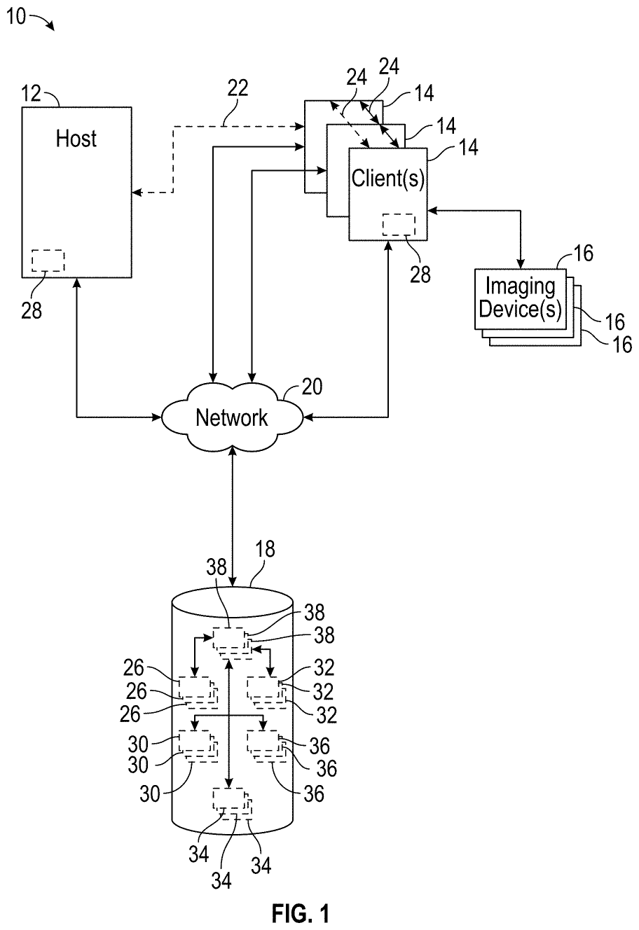

FIG. 1 illustrates an exemplary surgical planning system 10 (hereinafter referred to as “the system 10”). The system 10 may be used for planning orthopaedic procedures, including pre-operatively, intra-operatively, and/or post-operatively to create, edit, review, refine, and/or execute surgical plans. The system 10 may be utilized for various orthopaedic and other surgical procedures, such as an arthroplasty to repair a joint, for example.

Shoulder arthroplasty is periodically referenced throughout this disclosure to illustrate or emphasize certain features of the system 10. However, the teachings of this disclosure are not intended to be limited to any particular joint of the human musculoskeletal system and should therefore be understood as being applicable to the shoulder, knee, hip, ankle, wrist, etc. Moreover, the teachings of this disclosure are not intended to be limited to arthroplasty procedures and are therefore applicable to the repair of fractures and/or other deformities within the scope of this disclosure.

The system 10 may include, among other things, at least one host computer 12, one or more client computers 14, one or more imaging devices 16, a cloud-based storage system 18, and a network 20. The system 10 may include a greater or fewer number of subsystems within the scope of this disclosure.

The host computer 12 may be configured to execute one or more software programs. In some implementations, the host computer 12 may be more than one computer jointly configured to process software instructions serially or in parallel.

The host computer 12 may be operable to communicate with the network 20, which itself may include one or more computing devices. The network 20 may be a private local area network (LAN), a private wide area network (WAN), the Internet, or a mesh network, for example.

The host computer 12 and each client computer 14 may include one or more of a computer processor, memory, storage means, network devices and input and/or output devices and/or interfaces. The input devices may include a keyboard, mouse, etc. The output devices may include a monitor, speakers, printers, etc. The memory may, for example, include UVPROM, EEPROM, FLASH, RAM, ROM, DVD, CD, a hard drive, or other computer readable medium that may store data and/or other information relating to the surgical planning and implementation techniques disclosed herein. The host computer 12 and each client computer 14 may be a desktop computer, laptop computer, smart phone, tablet, virtual machine, or any other computing device. The interfaces may facilitate communication with the other systems and/or components of the network 20.

Each client computer 14 may be configured to communicate with the host computer 12 either directly, such as via a direct client interface 22, or over the network 20. In other implementations, the client computers 14 are configured to communicate with each other directly via a peer-to-peer interface 24.

Each client computer 14 may be operably coupled to one or more of the imaging devices 16. Each imaging device 16 may be configured to capture or acquire one or more images 26 of patient anatomy residing within a scan field (e.g., window) of the imaging device 16. The imaging device 16 may be configured to capture or acquire two dimensional (2D) and/or three dimensional (3D) greyscale and/or color images 26. Various imaging devices 16 may be utilized, including but not limited to an X-ray machine, a computerized tomography (CT) machine, or a magnetic resonance imaging (MRI) machine, for obtaining one or more images 26 of a patient. The images 26 may be saved to the storage system 18.

The client computers 14 may also be configured to execute one or more software programs, such as those associated with various surgical planning tools and/or applications. Each client computer 14 may be operable to access and locally and/or remotely execute a planning environment 28 for creating, editing, executing, refining, and/or reviewing one or more surgical plans 36 during pre-operative, intra-operative and/or post-operative phases of a surgery. The planning environment 28 may be a standalone software package or may be incorporated into another surgical tool. The planning environment 28 may be configured to communicate with the host computer 12 either over the network 20 or directly through the direct client interface 22.

The planning environment 28 may be further configured to interact with one or more of the imaging devices 16 to capture or acquire the images 26 of patient anatomy. The planning environment 28 may provide a display or visualization of one or more images 26, bone models 30, implant models 32, transfer models 34, and/or surgical plans 36 via one or more graphical user interfaces (GUI). Each image 26, bone model 30, implant model 32, transfer model 34, surgical plan 36, and other data and/or information may be stored on the storage system 18 in one or more files or records according to a specified data structure.

The planning environment 28 may include various modules for performing the desired planning functions. For example, as further discussed below, the planning environment 28 may include a data module for accessing, retrieving, and/or storing data concerning the surgical plans 36, a display module for displaying the data (e.g., within one or more GUIs), a spatial module for modifying the data displayed by the display module, and a comparison module for determining one or more relationships between selected bone models and selected implant models. However, a greater or fewer number of modules may be utilized, and/or one or more of the modules may be combined to provide the disclosed functionality.

The storage system 18 may be operable to store or otherwise provide data from/to other computing devices, such as the host computer 12 and/or the one or more client computers 14, of the system 10. The storage system 18 may be a storage area network device (SAN) configured to communicate with the host computer 12 and/or the client computers 14 over the network 20, for example. Although shown as a separate device of the system 10, the storage system 18 may in some implementations be incorporated within or directly coupled to the host computer 12 and/or client computers 14. The storage system 18 may be configured to store one or more of computer software instructions, data, database files, configuration information, etc.

[moss] In some implementations, the system 10 may be a client-server architecture configured to execute computer software on the host computer 12, which may be accessible by the client computers 14 using either a thin client application or a web browser that can be executed on the client computers 14. The host computer 12 may load the computer software instructions from local storage, or from the storage system 18, into memory and may execute the computer software using the one or more computer processors.

The system 10 may further include one or more databases 38. The databases 38 may be stored at a central location, such as on the storage system 18. In another implementation, one or more databases 38 may be stored at the host computer 12 and/or may be a distributed database provided by one or more of the client computers 14. Each database 38 may be a relational database configured to associate one or more images 26, bone models 30, implant models 32, and/or transfer models 34 to each other and/or to a respective surgical plan 36. Each surgical plan 36 may be associated with the anatomy of a respective patient. Each image 26, bone model 30, implant model 32, transfer model 34, and surgical plan 36 may be assigned a unique identifier or database entry for storage on the storage system 18. Each database 38 may be configured to store data and other information corresponding to the images 26, bone models 30, implant models 32, transfer models 34, and surgical plans 36 in one or more database records or entries, and/or may be configured to link or otherwise associate one or more files corresponding to each respective image 26, bone model 30, implant model 32, transfer model 34, and surgical plan 36. The various data stored in the database(s) 38 may correspond to respective patient anatomies from prior surgical cases, and may be arranged into one or more predefined categories such as sex, age, ethnicity, defect category, procedure type, anatomical makeup classification, surgeon, facility or organization, etc.

Each image 26 and bone model 30 may include data and other information obtained from one or more medical devices or tools, such as the imaging devices 16. The bone models 30 may include one or more digital images and/or coordinate information relating to an anatomy of the patient obtained or derived from image(s) 26 captured or otherwise obtained by the imaging device(s) 16.

Each implant model 32 and transfer model 34 may include coordinate information associated with a predefined design or a design established or modified by the planning environment 28. The predefined design may correspond to one or more components. The planning environment 28 may incorporate and/or interface with one or more modeling packages, such as a computer aided design (CAD) package, to render the models 30, 32, and 34 as two-dimensional (2D) and/or three-dimensional (3D) volumes or constructs, which may overlay one or more of the images 26 or bone models 30 in a display screen of one or more GUIs.

The implant models 32 may correspond to implants and components of various shapes and sizes. Each implant may include one or more components that may be situated at a surgical site including prosthetics, screws, anchors, grafts, etc. Each implant model 32 may correspond to a single component or may include two or more components that may be configured to establish an implant assembly. Each implant and associated component(s) may be formed of various materials, including metallic and/or non-metallic materials. Each bone model 30, implant model 32, and transfer model 34 may correspond to 2D and/or 3D geometry, and may be utilized to generate a wireframe, mesh, and/or solid construct in a GUI.

Each surgical plan 36 may be associated with or linked to one or more of the images 26, bone models 30, implant models 32, and/or transfer models 34. The surgical plan 36 may include various parameters associated with the images 26, bone models 30, implant models 32, and/or transfer models 34. For example, the surgical plan 36 may include parameters relating to the anatomical and planned implant centers of rotation associated with patient anatomy captured in the image(s) 26. The surgical plan 36 may further include parameters including spatial information relating to relative positioning and coordinate information of the selected bone model(s) 30, implant model(s) 32, and/or transfer model(s) 34.

The surgical plan 36 may define one or more revisions to a bone model 30 and information relating to a position of an implant model 32 and/or transfer model 34 relative to the original and/or revised bone model 30. The surgical plan 36 may include coordinate information relating to the revised bone model 30 and a relative position of the implant model 32 and/or transfer model 34 in one or more predefined data structure(s). The planning environment 28 may be configured to implement one or more revisions to the various models, either automatically or in response to user interaction with the user interface(s). Revisions to each bone model 30, implant model 32, transfer model 34, and/or surgical plan 36 may be stored in one or more of the databases 38, either automatically and/or in response to user interaction with the system 10.

One or more surgeons and/or other staff users may be presented with the planning environment 28 via the client computers 14 and may simultaneously access each image 26, bone model 30, implant model 32, transfer model 34, and surgical plan 36 stored in the database(s) 38. Each user may interact with the planning environment 28 to create, view, refine, and/or modify various aspects of the surgical plan 36. Each client computer 14 may be configured to store local instances of the images 26, bone models 30, implant models 32, transfer models 34, and/or surgical plans 36, which may be synchronized in real-time or periodically with the database(s) 38. The planning environment 28 may be a standalone software package executed on a client computer 14 or may be provided as one or more web-based services executed on the host computer 12, for example.

The system 10 described above may be configured for preoperatively planning surgical procedures. The preoperative planning provided by the system 10 may include, but is not limited to, features such as constructing a virtual model of a patient's anatomy, classifying the virtual model, identifying landmarks within the virtual model, selecting and orienting virtual implants within the virtual model, identifying and assessing center of rotation information within the virtual model, etc.

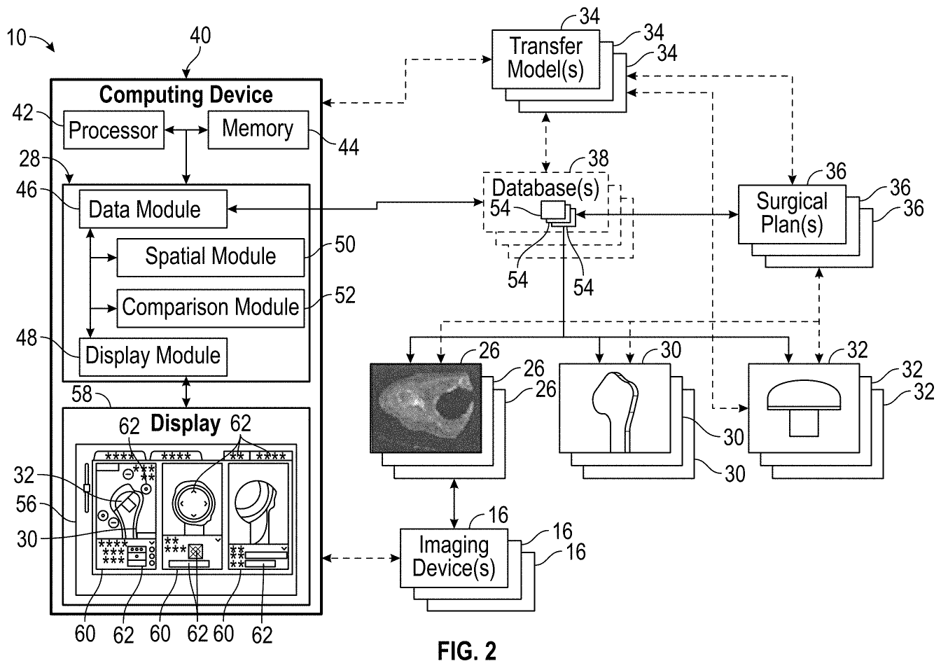

Referring now to FIG. 2, with continuing reference to FIG. 1, the system 10 may include a computing device 40 including at least one processor 42 coupled to a memory 44 that is capable of storing computer executable instructions. The computing device 40 may be considered representative of any of the computing devices disclosed herein, including but not limited to the host computer 12 and/or the client computers 14. The processor 42 may be configured to execute one or more of the planning environments 28 for creating, editing, executing, refining, and/or reviewing one or more surgical plans 36 and any associated bone models 30, implant models 32, and transfer models 34 during pre-operative, intra-operative, and/or post-operative phases of a surgery.

The processor 42 can be a custom made or commercially available processor, central processing unit (CPU), or generally any device for executing software instructions. The memory 44 can include any one or combination of volatile memory elements and/or nonvolatile memory elements. The processor 42 may be operably coupled to the memory 44 and may be configured to execute one or more programs stored in the memory 44 based on various inputs received from other devices or data sources associated with the system 10.

The planning environment 28 may include at least a data module 46, a display module 48, a spatial module 50, and a comparison module 52. Although four modules are shown in the highly schematic depiction of FIG. 2, it should be understood that a greater or fewer number of modules could be utilized, and/or further that one or more of the modules could be combined to provide the disclosed functionality for executing the planning environment 28.

The data module 46 may be configured to access, retrieve, and/or store data and other information in the database(s) 38 corresponding to one or more images 26 of patient anatomy, bone model(s) 30, implant model(s) 32, transfer model(s) 34, and/or surgical plan(s) 36. The data and other information may be stored in one or more databases 38 as one or more records or entries 54. In some implementations, the data and other information may be stored in one or more files that are accessible by referencing one or more objects or memory locations referenced by the entries 54.

The memory 44 may be configured to access, load, edit, and/or store instances of one or more images 26, bone models 30, implant models 32, transfer models 34, and/or surgical plans 36 in response to one or more commands from the data module 46. The data module 46 may be configured to cause the memory 44 to store a local instance of the image(s) 26, bone model(s) 30, implant model(s) 32, transfer model(s) 34, and/or surgical plan(s) 36, which may be synchronized with the entries 54 stored in the database(s) 38.

The data module 46 may be further configured to receive data and other information corresponding to at least one or more images 26 of patient anatomy from various sources, such as the imaging device(s) 16, for example. The data module 46 may be further configured to command the imaging device 16 to capture or acquire the images 26 automatically or in response to user interaction.

The display module 48 may be configured to display data and other information relating to one or more surgical plans 36 in at least one graphical user interface (GUI) 56, including one or more of the images 26, bone models 30, implant models 32, and/or transfer models 34. The computing device 40 may incorporate or be coupled to a display device 58. The display module 48 may be configured to allow the display device 58 to display information in the user interface 56. A surgeon, planning technician, or other user may interact with the user interface 56 within the planning environment 28 to view one or more images 26 of patient anatomy and/or any associated bone models 30, implant models 32, and transfer models 34. The surgeon, planning technician, or other user may interact with the user interface 56 via the planning environment 28 to create, edit, execute, refine, and/or review one or more surgical plans 36.

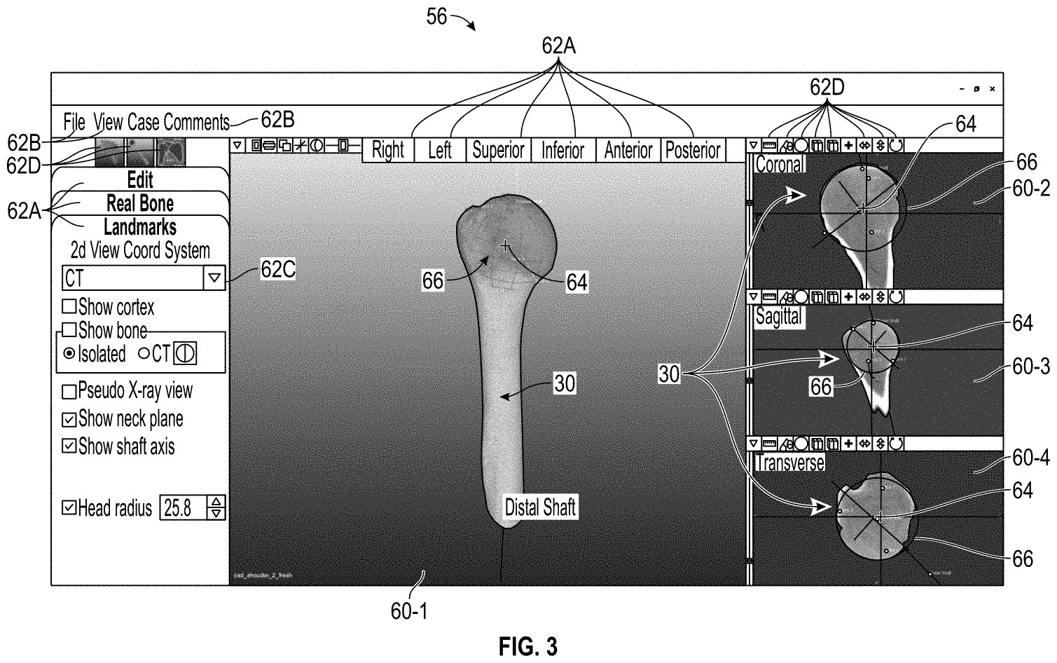

Referring to FIG. 3, with continuing reference to FIG. 2, the user interface 56 may include one or more display windows 60 and one or more objects 62 that may be presented within the display windows 60. The display windows 60 may include any number of windows, and the objects 62 may include any number of objects within the scope of this disclosure. A user, which in this embodiment may be a planning technician operating on the host computer 12, for example, may interact with the user interface 56, including the objects 62 and/or display windows 60, to retrieve, view, edit, store, etc., various aspects of a respective surgical plan 36, which may include information from the selected image(s) 26, bone model(s) 30, implant model(s) 32 and/or transfer model(s) 34.

The objects 62 may include graphics, such as menus, tabs, buttons, etc., that are accessible by user interaction and that may be organized in one or more menu items associated with the respective display windows 60. In an embodiment, the objects 62 include tabs 62A, drop-down menus 62B, drop-down lists 62C, buttons 62D, etc. Geometric objects, including bone model(s) 30 and/or other information relating to the surgical plan 36, may be displayed in one or more of the display windows 60.

The display windows 60 may include first, second, third, and fourth display windows 60-1, 60-2, 60-3, and 60-4. Although four display windows are illustrated in FIG. 3, it should be understood that a greater or fewer number of display windows 60 could be utilized in accordance with the teachings disclosed herein.

The first display window 60-1 may be associated with a three-dimensional (3D) view, and the second, third, and fourth windows 60-2, 60-3, and 60-4 may be associated with two-dimensional (2D) views. In an embodiment, the second, third, and fourth windows 60-2, 60-3, and 60-4 may be associated with two-dimensional (2D) DICOM views that can be presented to the user (e.g., coronal, sagittal, and transverse, respectively). The planning environment 28 may be configured such that changes in one of the display windows 60-1 to 60-4 are synchronized with each of the other display windows 60-1 to 60-4. The changes may be synchronized between the display windows 60-1 to 60-4 automatically and/or manually in response to user interaction

The display module 48 may be configured to display in the first, second, third, and fourth display windows 60-1, 60-2, 60-3, 60-4 a selected one of the bone models 30. The selected bone model 30 may correspond to a bone associated with a joint, such as a humerus as illustrated in FIG. 3. In the illustrated embodiment, the selected bone model 30 has already been created from the images 26 by segmenting, thresholding, and separating the bone from the joint, for example.

The spatial module 50 may be configured (such as via a desktop application executable by the processor 42, for example) to allow the user to identify various landmarks within the selected bone model 30. In an embodiment, a preoperative native anatomy center of rotation 64 may be identified within the anatomy associated with the selected bone model 30. In this disclosure, the term “preoperative native anatomy center of rotation” may be defined as the native center of rotation about which the joint mechanics of the joint associated with the selected bone model 30 will revolve. The preoperative native anatomy center of rotation 64 may be identified in a manner that ignores any joint erosion, and therefore the preoperative native anatomy center of rotation 64 may substantially mimic a center of rotation of the patient's original, non-deteriorated anatomy. The preoperative native anatomy center of rotation 64 may therefore be an approximation or interpolation of the patient's original, non-deteriorated anatomy.

For shoulder joints, the preoperative native anatomy center of rotation 64 may be defined relative to a humeral head of a humerus. For example, the user may, via the spatial module 50, position a sphere 66 within the 3D rendering of the selected bone model 30, which in this embodiment represents a patient's native humeral anatomy and is shown in the first display window 60-1. The sphere 66 may be placed visually utilizing both the 3D reconstruction of the selected bone model 30 within the first display window 60-1 and the 2D views provided within the second, third, and fourth display windows 60-2, 60-3, 60-4 and may be referenced relative to a landmark such as the scapular plane. The sphere 66 may be representative of the patient's native humeral head and may include a specific diameter and position relative to the scapular plane, and a center of the sphere 66 may thus be defined as the preoperative native anatomy center of rotation 64. The preoperative native anatomy center of rotation 64 may establish an X, Y, Z coordinate origin of 0,0,0 within the image data associated with the center of rotation feature.

Once identified, the location of the preoperative native anatomy center of rotation 64 may be saved as a landmark within the image data associated with the surgical plan 36 for the subject patient. The preoperative native anatomy center of rotation 64 may be subsequently referenced for further developing and refining the surgical plan 36 for the particular patient.

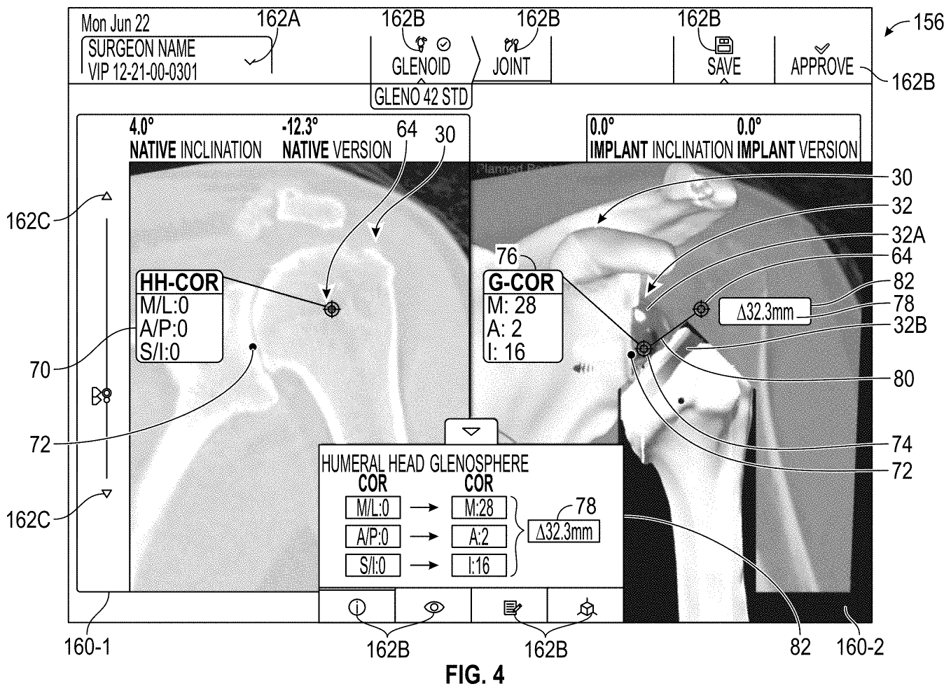

Referring now to FIG. 4, with continued reference to FIGS. 2 and 3, the display module 48 of the planning environment 28 may be further configured to display additional data/information relating to the center of rotation feature of the system 10 in another graphical user interface 156. A user, which in this embodiment may be a surgeon working on one of the client computers 14, may interact with the user interface 156 to retrieve, view, edit, store, etc. various aspects of the selected surgical plan 36.

The user interface 156 may include a first display window 160-1 and a second display window 160-2. Although two display windows are illustrated in FIG. 4, it should be understood that a greater or fewer number of display windows could be utilized in accordance with the teachings disclosed herein.

The user interface 156 may further include one or more objects 162 that allow the user to interact with the user interface 156, such as for specifying various aspects of the surgical plan 36. The objects 162 may include graphics, such as menus, tabs, buttons, etc., that are accessible by user interaction and that may be organized in one or more menu items associated with the respective display windows 160-1, 160-2. In an embodiment, the objects 162 include drop-down menus 162A, buttons 162B, and arrows 162C.

Geometric objects, including the selected bone model 30 and/or other information relating to the surgical plan 36, may be displayed in the display windows 160-1, 160-2. In an embodiment, the native preoperative anatomy associated with the selected bone model 30 may be presented within the display window 160-1, and one of the implant models 32 may be shown overlayed over the native anatomy associated with the selected bone model 30 in the display window 160-2. The display window 160-2 may therefore be configured to present planned postoperative aspects of the surgical plan 36. The display module 48 may be configured to display the selected bone model 30 in the display window 160-1 and may be further configured to display both the selected bone model 30 and the selected implant model 32 in the display window 160-2.

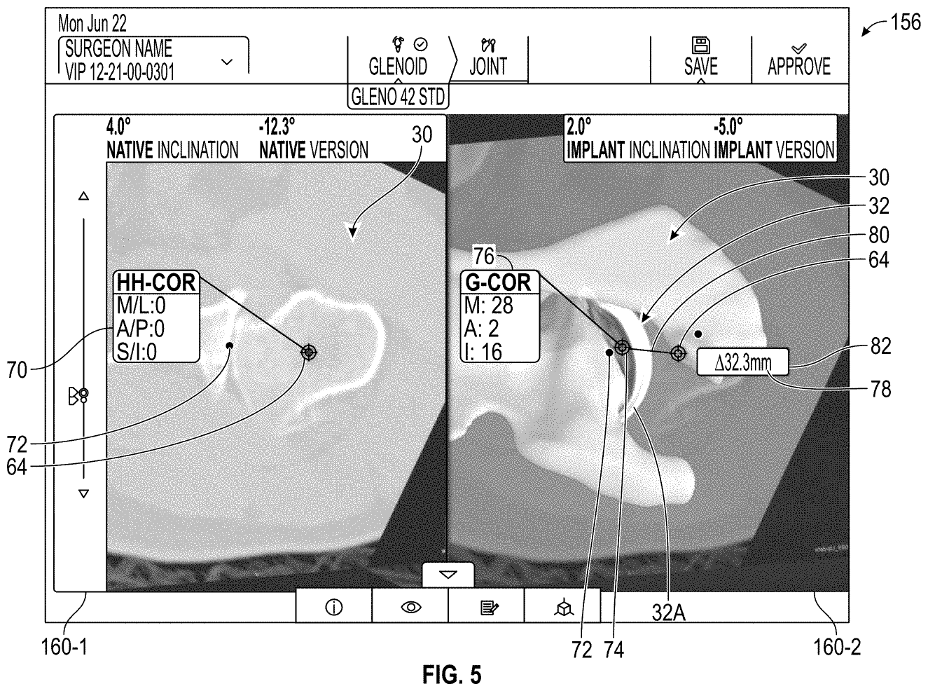

In the illustrated embodiment of FIG. 4, the display windows 160-1, 160-2 are both configured to illustrate coronal views of the various geometric objects. However, other views may be presented, such as transverse views (see FIG. 5), for example. The user may select one of the buttons 162B for toggling between the available views.

The display module 48 may be configured to display 3D representation(s) of the selected bone model 30 and the selected implant model 32 in the display window 160-2. The spatial module 50 may be configured to allow the user to interact with the display window 160-2, or another portion of the user interface 156, to move the selected bone model 30 and/or the selected implant model 32 in space (e.g., up, down, left, right). For example, the spatial module 50 may be configured to set a virtual position and/or a virtual axis in response to placement of a respective implant model 32 relative to the bone model 30 and associated patient anatomy. The virtual position and/or virtual axis may be set and/or adjusted automatically based on a position and orientation of the selected implant model 32 relative to the selected bone model 30 and/or in response to user interaction with the user interface 156. The user may select the components of the implant model 32 and their positions/orientations as part of the preoperative planning that can be performed within the user interface 156.

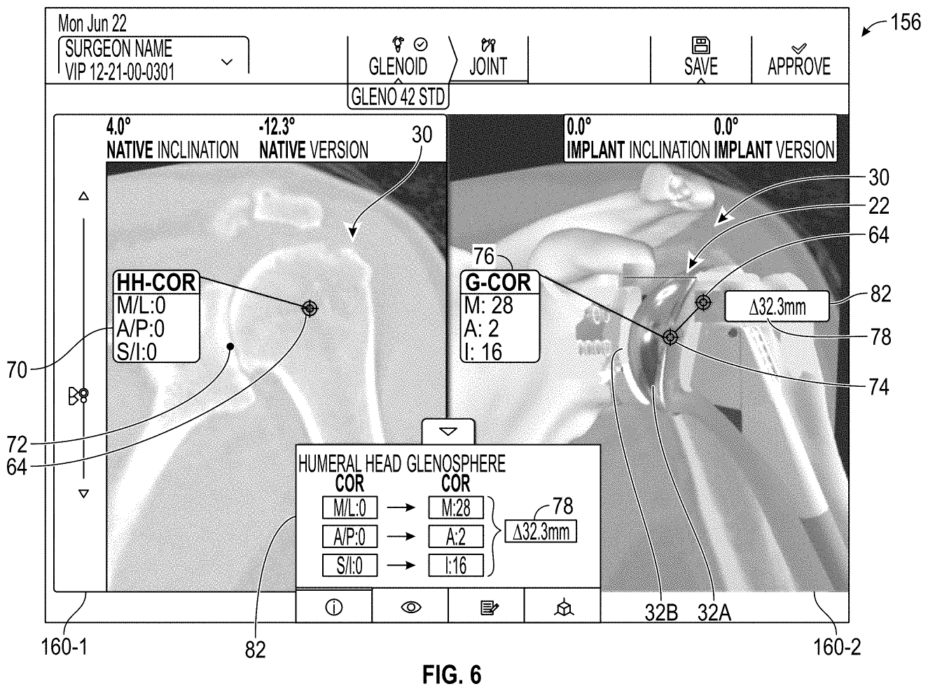

The selected implant model 32 may include one or more components. For example, the implant model 32 may include at least a first component 32A and a second component 32B coupled to the first component 32A to establish an assembly. The first component 32A may be configured to be at least partially received in a volume of the selected bone model 30. The second component 32B may have an articulation surface dimensioned to mate with an articular surface of an opposed bone or implant. In the illustrated embodiment of FIG. 4, the selected implant model 32 is a reverse total shoulder arthroplasty implant assembly. However, other configurations are also contemplated, including but not limited to, anatomical total shoulder arthroplasty implant assemblies (see, for example, the implementation of FIG. 6).

The display module 48 may be configured to display a sectional view of the selected implant model 32 and/or the selected bone model 30 in one or both of the display windows 160-1, 160-2. The sectional views may be presented as an image of the bone associated with the selected bone model 30. An orientation of the sectional view may be predefined or may be specified in response to user interaction with the user interface 156, such as by pressing the arrows 162C, for example.

The display module 48 may be further configured to display the preoperative native anatomy center of rotation 64 within one or both of the display windows 160-1, 160-2. The coordinates (0, 0, 0) associated with the preoperative native anatomy center of rotation 64 may be displayed in a graphic indicator 70. The graphic indicator 70 may overlay or be arranged adjacent to the display window 160-1, for example.

The coordinates associated with the preoperative native anatomy center of rotation 64 may be displayed in anatomical terms. For example, the first coordinate may be a medial or lateral location of the center of rotation value, the second coordinate may be an anterior or posterior location of the center of rotation value, and the third coordinate may be a superior or inferior location of the center of rotation value.

A native center of rotation 72 of another bone (e.g., a glenoid) of the selected bone model 30 may also displayed, either with or without anatomical coordinates, for reference in the display window 160-1. These values and anatomical locations are native values that may be derived from landmarking procedures performed by a planning technician at the host computer 12. In some implementations, the native centers of rotation 64, 72 are native landmarks and thus their associated values cannot be adjusted by the surgeon user within the user interface 156.

The spatial module 50 may be configured to identify a planned postoperative implant center of rotation 74 of the first component 32A of the selected implant model 32. The planned postoperative implant center of rotation 74 may be automatically updated as the user manipulates the selected implant model 32 relative to the bone model 30 within the display window 160-2. In implementations in which the subject surgical plan 36 relates to a reverse total shoulder arthroplasty procedure (see, e.g., FIGS. 4-5), the first component 32A of the selected implant model 32 is a glenosphere implant and the planned postoperative implant center of rotation 74 is the center of the glenosphere implant. In other implementations in which the subject surgical plan 36 relates to an anatomical total shoulder arthroplasty procedure (see, e.g., FIG. 6), the first component 32A of the selected implant model 32 is a humeral head implant and the planned postoperative implant center of rotation 74 is the center of the humeral head implant.

Coordinates associated with the planned postoperative implant center of rotation 74 may be displayed as corresponding anatomical coordinates (in millimeter units, for example) relative to the preoperative native anatomy center of rotation 64. For example, the first coordinate may be a medial or lateral coordinate of the center of rotation value of the implant relative to the medial or lateral coordinate of the preoperative native anatomy center of rotation 64, the second coordinate may be an anterior or posterior coordinate of the center of rotation value of the implant relative to the anterior or posterior coordinate of the preoperative native anatomy center of rotation 64, and the third coordinate may be a superior or inferior coordinate of the center of rotation value of the implant relative to the superior or inferior coordinate of the preoperative native anatomy center of rotation 64. The anatomical direction for each of these coordinates may be based on a resampling of imaging data sets relative to a landmark (e.g., a scapular plane) saved within the bone model 30.

The spatial module 50 may be configured to cause the display module 48 to display the coordinates associated with the planned postoperative implant center of rotation 74 in a graphic indicator 76. The graphic indicator 76 may overlay or be arranged adjacent to the display window 160-2, for example.

The comparison module 52 may be configured to generate or set one or more parameters associated with implementing the surgical plan 36. The parameters may include one or more settings or dimensions associated with center of rotation data derived from a positioning of the implant model 32 relative to the bone model 30, for example.

In an embodiment, the comparison module 52 may be configured to derive a delta distance 78 between the planned postoperative implant center of rotation 74 and the preoperative native anatomy center of rotation 64. The delta distance 78 is essentially the length of a vector in the three dimensional space between the planned postoperative implant center of rotation 74 and the preoperative native anatomy center of rotation 64 and thus can be calculated using the three anatomical coordinates associated with the planned postoperative implant center of rotation 74. More particularly, the delta distance 78 may be calculated by calculating the square root of the sum of the squared values of each of the medial/lateral coordinate, the anterior/posterior coordinate, and the superior/inferior coordinate of the planned postoperative implant center of rotation 74. This calculation may be represented by the following equation:

Δ=√(M/L²+A/P²+S/I²)

The comparison module 52 may be further configured to cause the display module 48 to display the delta distance 78 within the user interface 156. In an embodiment, the delta distance 78 may be visually indicated by a line 80 drawn between the implant center of rotation 74 and the preoperative native anatomy center of rotation 64. In another embodiment, the actual value (e.g., in millimeters) of the delta distance 78 may be displayed within one or more graphic indicators 82. The graphic indicators 82 may overlay or be arranged adjacent to the display window 160-2, for example.

Using the user interface 156, the user may select the implant components of the implant model 32 and their positions/orientations relative to the bone model 30 during preoperative planning. These selections may be utilized to derive the location of the planned postoperative implant center of rotation 74 and the value of the delta distance 78. These values may then be assessed by the user to adjust the planned implant type, size, position, orientation, etc. to achieve a specific postoperative center of rotation or delta distance most appropriate to the patient's anatomy. In general, it is believed that smaller delta distances are likely to provide improved surgical outcomes compared to larger delta distances for anatomic procedures.

The display module 48 may be further configured to provide an indication of subluxation percentage to the user within the user interface 156 or another user interface. For example, the display module 48 may provide a volumetric percentage of the humeral head sphere 66 that is positioned posterior to a scapular plane as a 2D circle and the scapular plane as a line overlayed on the imaging data.

Once a satisfactory delta distance 78 has been achieved, the user may save the surgical plan 36. The surgical plan 36 may be saved to the appropriate database 38 of the storage system 18 and approved by pressing one or more of the buttons 162B (e.g., the save button and/or the approve button). The saved and approved surgical plan 36 may include various parameters associated with the subject patient including both the planned postoperative implant center of rotation 74 and the value of the delta distance 78. As further discussed below, these parameters may be used for tracking/comparing patient outcome data and anatomical makeup information.

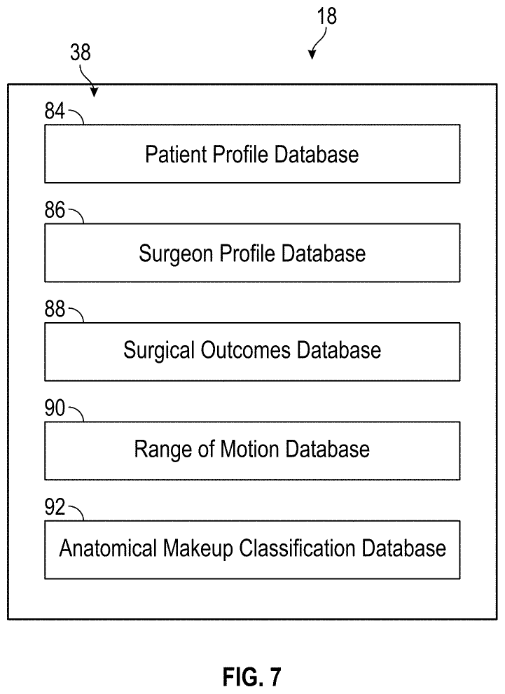

Referring now to FIG. 7, with continued reference to FIGS. 2-6, the computing device 40 of the system 10 may interface with the storage system 18 over the network 20 for accessing various databases 38 stored thereon in order to establish and implement the surgical plans 36. The databases 38 of the storage system 18 may include a patient profile database 84, a surgeon profile database 86, a surgical outcomes database 88, a range of motion database 90, and an anatomical makeup classification database 92. Additional databases could be stored on and accessed from the storage system 18 within the scope of this disclosure. Moreover, although shown as separate databases, one or more of the databases could be combined or linked together. For example, the anatomical makeup classification database 92 could be combined or linked with one or more of the patient profile database 84, the surgical outcomes database 88, and the range of motion database 90.

The patient profile database 84 may include information that is part of an indexed and stored record or entry related to one or more current patients associated with the system 10. The information stored on the patient profile database 84 may include the sex, age, ethnicity, height, weight, defect category, procedure type, surgeon, facility or organization, dominant joint, acts of daily living/lifestyle goals profile (e.g., desired post-surgery range of motion for abduction, adduction, external rotation, internal rotation, extension, flexion, external rotation combined with 60° abduction, internal rotation with 60° abduction, etc.), current surgical plan information including saved planned postoperative implant center of rotation 74 and the delta distance 78, etc. for each patient. The patient profile database 84 may further store or link to the images 26 for a given patient, including images 26 that include landmark identifiers of the preoperative native anatomy center of rotation 64 and the planned postoperative implant center of rotation 74.

[mom] The surgeon profile database 86 may include information that is part of indexed and stored records or entries related to one or more surgeon users associated with the system 10. The information stored on the surgeon profile database 86 may include the surgeon's name, facility or organization, historical data concerning the types of prior surgeries planned by the surgeon using the system 10, data concerning the types of implants included in the surgeon's preoperative surgical plans, data concerning the actual implants utilized in the surgeon's prior surgeries, data regarding the delta distances between preoperative native anatomy centers of rotation and planned postoperative implant centers of rotation in the surgeon's prior surgeries, etc. In some implementations, the surgeon profile database 86 may interface with the patient profile database 84 for linking each surgeon from the surgeon profile database 86 to his/her patients listed in the patient profile database 84.

The surgical outcomes database 88 may include information that is part of indexed and stored records or entries related to one or more prior patients associated with the system 10. The surgical outcomes database 88 may be created based on information logged by surgeons and/or other staff users after performing each surgery and at each follow-up visit for indicating the progress of the prior patient. The information stored on the surgical outcomes database 88 may include the sex, age, ethnicity, height, weight, defect category, procedure type, specific implants used, surgeon, facility or organization, dominant joint, visual analog pain scores, ASES scores, achieved acts of daily living/lifestyle profile (e.g., achieved post-surgery range of motion for abduction, adduction, external rotation, internal rotation, extension, flexion, external rotation combined with 60° abduction, internal rotation with 60° abduction, etc.), surgical plan information including planned postoperative implant center of rotation and the delta distance between the preoperative native anatomy center of rotation and the planned postoperative implant center of rotation, etc. for each prior patient. The surgical outcomes database 88 may additionally store or link to preoperative and postoperative images 26 for each prior patient.

The range of motion database 90 may include information that is part of indexed and stored records or entries related to one or more current and prior patients associated with the system 10. The range of motion database 90 may store range of motion data derived from range of motion simulations performed by the computing device 40 for each surgical plan 36. The range of motion data may include information related to simulated joint motions (e.g., abduction/adduction, flexion/extension, internal/external rotation, etc.), identified contact or collision points for various implant positions, angular arc and mode of collision (e.g., implant-to-implant, implant-to-bone, bone-to-bone, etc.) for various implant positions, adjusted centers of rotation of implants in multiple increments and offset directions for various implant positions, etc. The range of motion database 90 may additionally store the delta distance between the preoperative native anatomy center of rotation and the planned postoperative implant center of rotation for each of the adjusted centers of rotation.

The anatomical makeup classification database 92 may store a plurality of anatomical makeup classifications that characterize anatomical differences and variances within the anatomical differences within a representative patient population for one or more intended surgeries (e.g., anatomical total shoulder, reverse shoulder, etc.). In some implementations, the representative patient population may be derived by analyzing image data, such as images from the prior patients stored on the surgical outcomes database 88 and/or any other imaging source, associated with a plurality of prior patients who have already received the intended surgery. Each of the plurality of anatomical makeup classifications is a numerical classification of an anatomical makeup of a bone or a joint of the representative patient population.

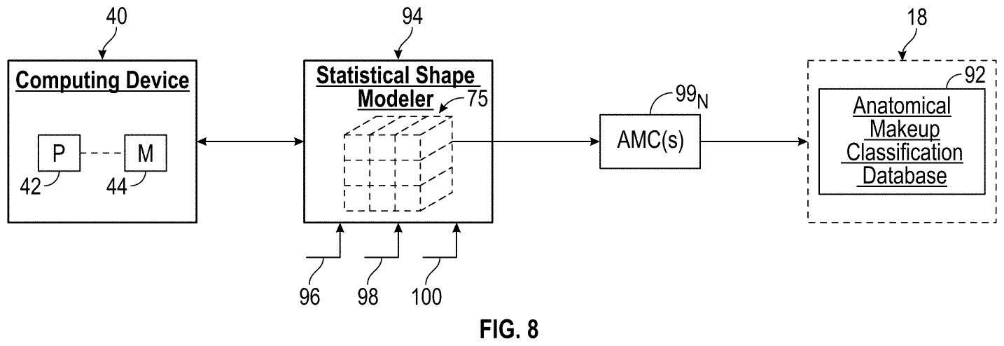

Referring now to FIG. 8, a statistical shape modeler 94 may be utilized to create the anatomical makeup classification database 92. The statistical shape modeler 94 may be a software package stored in the memory 44 of the computing device 40 or in the storage system 18 and which may be executed by the processor 42. The statistical shape modeler 94 may receive a plurality of sets of image data 96 associated with a bone or joint of interest. In some implementations, the sets of image data 96 is made up of tens of thousands of sets of image data. Each set of image data 96 may include 2D and/or 3D anatomical images specific to prior patients of a representative patient population for the bone or joint of interest and related to a given type of surgery. The statistical shape modeler 94 may analyze the plurality of sets of image data 96 for constructing a statistical shape model 95.

As an input, the statistical shape modeler 94 may receive a plurality of predefined modes 98 to be used for analyzing the plurality of sets of image data 96. Each of the modes 98 is a descriptor configured for characterizing anatomical differences in the bone or joint associated with the statistical shape model 95. Exemplary modes 98 that may be provided to the statistical shape modeler 94 may include but are not limited to size of glenoid, size of scapula, amount of inclination, amount of version, projected amount of glenoid and sagittal neck length, angle of glenoid relative to scapular neck, critical shoulder angle, projection of acromion and/or coracoid, size of humeral head, varus/valgus of humeral head, varus/valgus of femur and/or tibia, internal/external rotation of femur and/or tibia, integrity of subscapularis, deltoid, and/or supraspinatus, ML and AP width, intercondylar notch depth, tibial slope, Q-angle of the knee, ACL/PCL stability, MCL/LCL stability, amount of flexion, amount of extension, quality and amount of soft tissue surrounding joint, patellar tracking angle, bone density, bone quality subluxation percentage, anatomical landmarks, joint space, pre-operative range of motion, delta distance between preoperative native anatomy center of rotation and planned postoperative implant center of rotation, any combinations of the foregoing, etc.

In some implementations, at least seven different modes may be utilized by the statistical shape modeler 94 to characterize the statistical shape model 95. However, a greater or fewer number of modes may be provided within the scope of this disclosure.

In some implementations, the modes 98 may not be predefined. Rather, the statistical shape modeler 94 may be programmed to utilize artificial intelligence (e.g. a neural network) or machine learning to extrapolate the modes that best relate to the bone or joint being modeled within the statistical shape model 95.

As another input, the statistical shape modeler 94 may receive a plurality of predefined standard deviations 100 to be used for analyzing the plurality of sets of image data 96. Each standard deviation 100 may represent anatomical variances (e.g., distances between features, orientation of features, relative features, etc.) contained within each of the plurality of predefined modes 98. The standard deviations 100 may be used to validate a percentile coverage of the representative patient population that is represented within the statistical shape model 95. In some implementations, at least seven different standards of deviation (e.g., −3, −2, −1, 0, 1, 2, and 3) may be utilized by the statistical shape modeler 94 to further characterize all anatomical variances contained within the anatomies described within the statistical shape model 95. However, a greater or fewer number of standard deviations could be utilized within the scope of this disclosure.

The statistical shape modeler 94 may, in response to commands from the processor 42, combine the plurality of standard deviations 100 with the plurality of predefined modes 98 to assign a plurality of anatomical makeup classifications 99_{N}, wherein N is any number, to the bone or joint associated with the statistical shape model 95 in order to categorize the anatomical makeup of the entire patient population represented within the statistical shape model 95. Each anatomical makeup classification 99_{N} may then be saved in the anatomical makeup classification database 92 of the storage system 18.

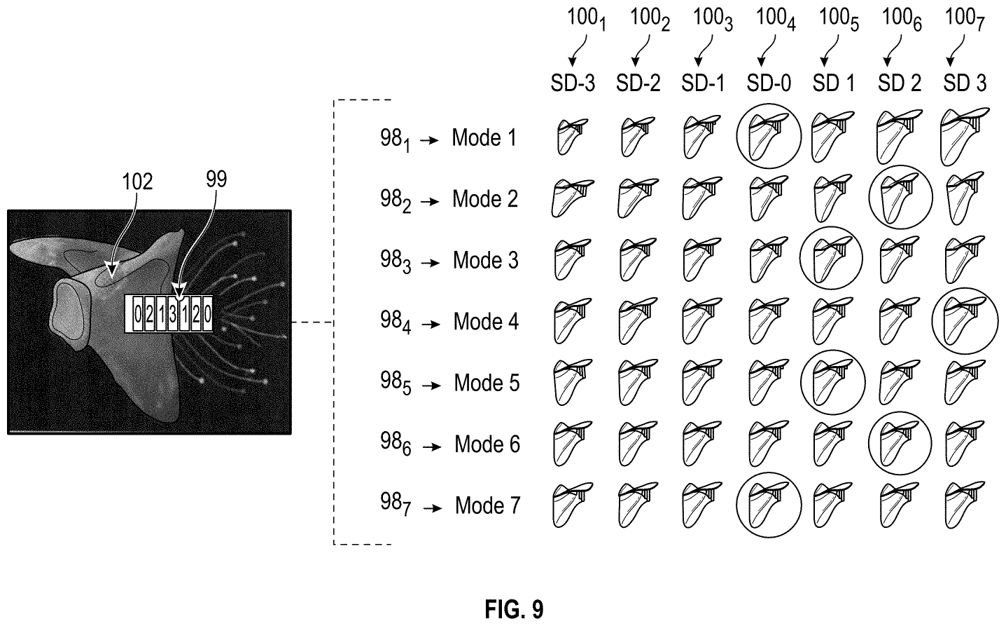

FIG. 9 schematically depicts an exemplary anatomic makeup classification 99 as assigned to a specific bone model 102 derived from the statistical shape model 95. In an embodiment, the bone model 102 is a 3D model of a scapula of a shoulder joint. However, other bones and joints could also be classified in a similar manner.

The statistical shape modeler 94 of FIG. 8 may analyze the bone model 102 in respect to each of a plurality of modes 98₁ to 98₇, in order to characterize any anatomical differences in the bone model 102 compared to the other similar bones/joints associated with the statistical shape model 95. Of course, a greater or fewer number of modes are possible.

The statistical shape modeler 94 may further characterize any anatomical variances contained within each of the plurality of predefined modes 98₁-98₇ by analyzing each of the modes with respect to a plurality of standard deviations 100₁-100₇. Of course, a greater or fewer number of standards of deviation are possible.

In the implementation shown in FIG. 9, the bone model 102 has been assigned the numerical value 0213120 as its anatomical makeup classification 99. This numerical value represents a standard of deviation of 0 within the first mode 98₁, a standard of deviation of 2 within the second mode 98₂, a standard of deviation of 1 within the third mode 98₃, a standard of deviation of 3 within the fourth mode 98₄, a standard of deviation of 1 in the fifth mode 985, a standard of deviation of 2 within the sixth mode 986, and a standard of deviation of 0 in the seventh mode 98₇. The anatomical makeup classification 99 is thus a unique numeric identifier for describing the anatomy associated with the bone model 102.

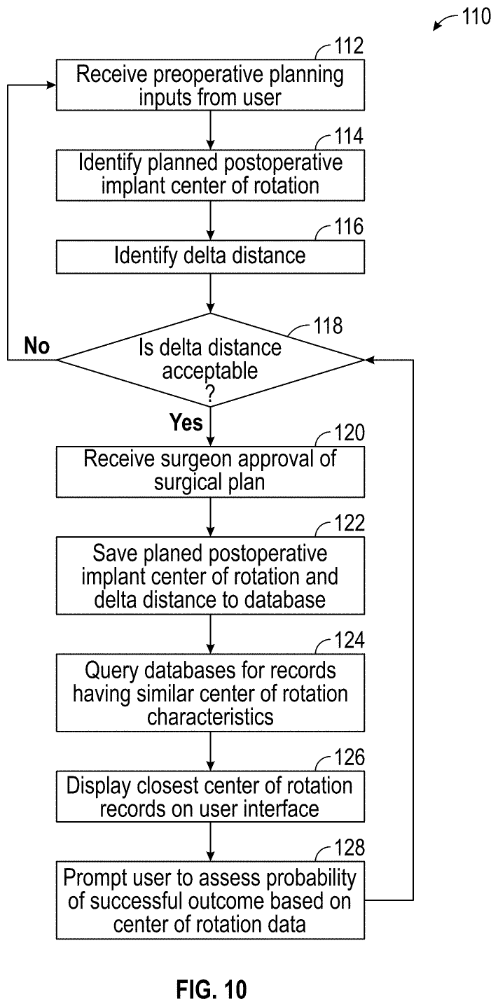

FIG. 10, with continued reference to FIGS. 1-9, schematically illustrates a method 110 for planning an orthopedic procedure for a respective patient using the system 10. The method 110 may be performed by a user (e.g., a surgeon) as part of a surgical planning procedure for preparing a surgical plan for the patient. Fewer or additional steps than are recited below could be performed within the scope of this disclosure, and the recited order of steps is not intended to limit this disclosure.

The system 10, via any of its associated computing devices and modules, may be configured to execute each of the steps of the method 110. In an exemplary implementation, the computing device 40 of one or more of the client computers 14 may be programmed to execute the method 110. The method 110 therefore assumes that a planning technician has already created a bone model 30 of the patient's anatomy and identified the preoperative native anatomy center of rotation 64 within the bone model 30. However, other implementations are further contemplated within the scope of this disclosure.

Preoperative planning inputs may be received from the user at step 112. The preoperative planning inputs may include implant type selection, implant size selection, implant location, implant orientation, implant offset, etc.

Based on the preoperative planning inputs, the planned postoperative implant center of rotation 74 may be identified at step 114. The delta distance 78 between the planned postoperative implant center of rotation 74 and the preoperative native anatomy center of rotation 64 may then be identified at step 116.

At step 118, the user may be prompted to indicate whether the identified center of rotation delta distance 78 is acceptable. If NO, the method 110 may return to step 112 for receiving additional user modifications to inputs such as implant type, size, location, orientation, etc.

The method 110 may proceed to step 120 when the delta distance 78 is acceptable to the user. At this step, the system 10 may receive approval of the preoperative surgical plan from the user. The planned postoperative implant center of rotation 74 and the delta distance 78 associated with the approved surgical plan are then saved in the appropriate database(s) of the storage system 18 at step 122.

Next, at step 124, the computing device 40 may query the anatomical makeup classification database 92 and/or the surgical outcomes database 88 to locate records stored therein that have similar anatomical makeup classifications and similar center of rotation characteristics (e.g., preoperative native anatomy center of rotation, planned implant center of rotation, delta distance, etc.). The records having the closest center of rotation characteristics may be displayed on a user interface of the computing device 40 at step 126.

The user may be prompted to assess the probability of a successful surgical outcome based on the center or rotation data at block 128. The method 110 may then return to step 118 by querying the user to again indicate whether the center of rotation delta distance 78 is acceptable. The method 110 may then continue in a looped fashion until the user no longer makes any further modifications to the implant type, size, location, orientation, etc.

The proposed surgical planning systems and methods of this disclosure may be utilized to create and implement surgical plans that are tailored to the individual patient, which may improve healing. The disclosed systems and methods may preoperatively analyze center or rotation data, including the delta distance between the planned implant center of rotation and the preoperative native anatomy center of rotation, in order to better assess the probability of obtaining successful surgical outcomes. The proposed systems and methods therefore provide improved functionality compared to prior planning systems.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A surgical planning system, comprising:
a memory device configured to store computer executable instructions; and
a processor operably coupled to the memory device and configured to execute the computer executable instructions to:
receive an input related to a position of an implant model relative to a bone model within a surgical planning environment; and
calculate a deviation between a planned postoperative implant center of rotation of the implant model and a preoperative native anatomy center of rotation of the bone model.

2. The surgical planning system as recited in claim 1, wherein the preoperative native anatomy center of rotation is the native center of rotation about which a joint mechanics of a joint associated with the bone model will revolve.

3. The surgical planning system as recited in claim 2, wherein the preoperative native anatomy center of rotation is an interpolation of an original, non-deteriorated anatomy of a patient associated with the bone model.

4. The surgical planning system as recited in claim 1, wherein the planned postoperative implant center of rotation is a value associated with an implant associated with the implant model.

5. The surgical planning system as recited in claim 4, wherein the implant is a glenosphere implant.

6. The surgical planning system as recited in claim 4, wherein the implant is a humeral head implant.

7. The surgical planning system as recited in claim 1, wherein the deviation is a delta distance representing a length of a vector in a three dimensional space between the planned postoperative implant center of rotation and the preoperative native anatomy center of rotation.

8. The surgical planning system as recited in claim 7, wherein the processor is further configured to cause the delta distance to be displayed within a user interface of the surgical planning environment.

9. The surgical planning system as recited in claim 8, wherein the delta distance is visually indicated by a line extending between the planned postoperative implant center of rotation and the preoperative native anatomy center of rotation.

10. The surgical planning system as recited in claim 8, wherein the delta distance is visually indicated by a numerical value in a graphic indicator of the user interface.

11. The surgical planning system as recited in claim 1, wherein the processor is further configured to query a database of the surgical planning system for records having similar center of rotation characteristics.

12. The surgical planning system as recited in claim 11, wherein the database is an anatomical makeup classification database that stores a plurality of anatomical makeup classifications that characterize anatomical differences and variances within the anatomical differences within a representative patient population.

13. The surgical planning system as recited in claim 12, wherein each of the plurality of anatomical makeup classifications is a numerical classification of an anatomical makeup of a bone or a joint of the representative patient population.

14. The surgical planning system as recited in claim 11, wherein the processor is further configured to command that a user be prompted to assess a probability of a successful surgical outcome based on the records having the similar center of rotation characteristics.

15. A surgical planning system, comprising:
a memory device configured to store computer executable instructions; and
a processor operably coupled to the memory device and configured to execute the computer executable instructions to execute a surgical planning environment that includes a display module, a spatial module, and a comparison module,
wherein the memory device is configured to store an implant model and a bone model,
wherein the bone model includes an identification of a preoperative native anatomy center of rotation,
wherein the spatial module is configured to establish a planned postoperative implant center of rotation of an implant of the implant model that is overlayed on the bone model,
wherein the comparison module is configured to determine a delta distance between the planned postoperative implant center of rotation and the preoperative native anatomy center of rotation,
wherein the display module is configured to display the delta distance in a display window of a graphical user interface.

16. The surgical planning system as recited in claim 15, wherein the delta distance is visually indicated by a line extending between the planned postoperative implant center of rotation and the preoperative native anatomy center of rotation.

17. The surgical planning system as recited in claim 15, wherein the delta distance is visually indicated by a numerical value in the display window.

18. The surgical planning system as recited in claim 15, wherein the preoperative native anatomy center of rotation is an interpolation of an original, non-deteriorated anatomy of a patient associated with the bone model.

19. The surgical planning system as recited in claim 15, wherein the planned postoperative implant center of rotation is a value associated with the implant, and further wherein the implant is a glenosphere implant or a humeral head implant.

20. A computer implemented surgical planning method comprising the steps of:
receiving a preoperative planning input from a user,
wherein the preoperative planning input includes a position of an implant model relative to a bone model of a subject patient;
identifying a planned postoperative implant center of rotation of an implant of the implant model relative to the bone model; and
calculating a delta distance between the planned postoperative implant center of rotation and a preoperative native anatomy center of rotation of a bone of the bone model.
